(19) United States
(12) Patent Application Publication
(10) Pub. No.: US 2023/0042375 A1
(43) Pub. Date: Feb. 9, 2023

(54) MEMBRANE BASED CHEMILUMINESCENCE IMMUNOCHROMATOGRAPHY ASSAY AND ITS USE

(71) Applicant: Marvin LIU, Los Angeles, CA (US)

(72) Inventor: Marvin LIU, Los Angeles, CA (US)

(21) Appl. No.: 17/949,157

(22) Filed: Sep. 20, 2022

(51) Int. Cl. G01N 33/543; G01N 33/72

(52) U.S. Cl. CPC G01N 33/54393; G01N 33/72; G01N 33/54388; G01N 2470/06

(57) ABSTRACT

A chemiluminescence immunochromatographic detection assay, comprising a solid membrane, a capture agent, a chemiluminescent conjugate, a testing buffer, a chemiluminescent reaction solution and a chemiluminescent reader. The capture agent is coated on the solid membrane, the chemiluminescent flows through the solid membrane and absorbed in a water absorbent structure, and the target analyte is captured and immobilized by capture agent on the solid membrane, and the uncapped chemiluminescent conjugate is cleaned up by testing buffer through the solid membrane, The complex of chemiluminescent conjugate and target analyte be immobilized on the solid membrane and placed for the quantitative detection of the light by the chemiluminescent reaction solution and the chemiluminescent reader, and complete the quantitative detection. This technology is suitable for chemiluminescent immunochromatographic detection of various analyte immune analysis, and is characterized as high efficiency, convenience, accuracy and high speed in important clinical application.

### BACKGROUND OF THE INVENTION

### Field of the Invention

The present invention relates to the technical field of medical devices, in particular to a membrane based chemiluminescence immunochromatographic detection assay and its use.

### Description of the Related Art

Immunoassays have been widely used to determine the presence or concentration of a macromolecule or a small molecule in a solution through the use of an antibody or an antigen for medical and research purposes, as well as environmental, drug, food and industrial analysis, which are based on the highly specific binding between an antigen and an antibody. The molecule detected by the immunoassay is referred to as an analyte, also known as an antigen, and is a protein in many cases, and has the ability of an antibody to be recognized and bind specifically, in what might be a complex mixture of macromolecules. Aside from the binding of an antibody to its analyte, the means to produce a measurable signal in response to the binding is another very important feature of the immunoassays including emitting radiation, changing color, fluorescing under light, or induce to emit light. Commonly used immunoassays include immunoturbidimetry, immunochemiluminescence, immunofluorescence, enzyme-linked immunosorbent assays (ELISA), colloidal gold immunochromatography, fluorescence immunochromatography, and latex immunochromatography, which offers simple, rapid, highly sensitive, and easy-to-use methods for routine analyses in immune detection.

Immunoturbidimetry is used to determine the soluble complex of an antibody and an antigen, in which an antigen-antibody complex aggregates to form particles that can be optically detected by a photometer, and a measurement is then given for the amount of light absorbed to calculate the concentration of an analyte in a solution.

Immunochromatography, also known as a lateral flow test, is a simple and fast assay intended to detect the presence or concentration of an analyte in a liquid sample through a solid, membrane-based reaction. It is widely used in medical diagnostics at home, point of care, and in the laboratory. Antibody-antibody sandwich one-step assay is used in this detection. The measurable labels used in this detection include colloidal gold, fluorescent dyes, fluorescent microsphere, colored latex particles, and magnetic nanobeads. The antibody-labels complex is used as a detector and is coated on the conjugate pad, and the paired unconjugated antibody is used as the capture and coated on the solid membrane. When a sample is loaded on the conjugate pad, the analyte in the sample specifically binds to the antibody that is conjugated with labels, forms an antigen-antibody label complex, and then the complex migrates on the solid membrane and is captured by the paired unconjugated antibody and immobilized on the solid membrane, and then a measurable band appears on the detection line for further measurement. Characteristics of this technology are that it is simple, rapid, sensitive, low cost, and easy to operate.

Chemiluminescent immunoassay (CLIA) is an immunoassay technique where the measurable label is a luminescent molecule that induces light emission. It is the most widely used immunoassay in clinical detection, which includes a direct assay, using luminophore labels, or indirect assay using enzyme labels. In a direct assay, the luminophore labels used are acridinium and ruthenium esters, while the enzymatic labels used in indirect methods are alkaline phosphatase with adamantyl 1, 2-dioxetane arylphosphate (AMPPD) substrate and horseradish peroxidase with luminol or its derivatives as substrate. These synthesizing molecules and the addition of an enhancer can further boost the light emission to a highly elevated analytic sensitivity (mol-16 per litre) in magnetic beads based multiplexed immunoassay system, which is much superior to that attainable by other immunoassay methods as RIA, ELISA, immunochromatography and fluoroimmunoenzymatic (FEIA) methods. The tubular magnetic particle-based chemiluminescence immunoassay has been the most commonly used assay for detection. However, there are some limitations to its application including limited detection capacity, high costs, limited testing panels, closed analytical systems, and reagent cold chain transportation.

Therefore, establishment of a chemiluminescence immunoassay combined with immunochromatography will be more simple, costeffective, rapid, and easy to use. However, the high-noise background and a lack of effective linear detection range are the most immediate technical problems to be solved for this combination. So it is important to find a way to establish a chemiluminescent immunochromatogranphic assay with low noise background and high linear detection range for further application.

### SUMMARY OF THE INVENTION

The objective of the present invention is to provide a solution to a major technical problem that cannot solved by current technology, which is that linear range of detection is too narrow and is not reached throughout the entire assay, leading to inaccurate test results. This problem could be solved with membrane based chemiluminescence immunochromatographic assay, which broadens the linear range of detection and is characterized by high sensitivity, fast detection, low cost, and an easy-to-use design.

The present invention provides a membrane based chemiluminescence immunochromatographic detection assay, comprising: a solid membrane, a capture agent, a chemiluminescent label, a chemiluminescent conjugate, a testing buffer, a chemiluminescence reaction solution, and a chemiluminescence reader, wherein the capture agent is coated on the solid membrane in a scattered, distributed manner, and the coating area covered by the capture agent on the solid membrane is positively related to the linear detection range of the chemiluminescent immunochromatographic detection.

The membrane based chemiluminescence immunochromatographic detection assay further provides the solid membrane, the capture agent, the chemiluminescent label, the chemiluminescent conjugate, the testing buffer, the chemiluminescence reaction solution, and the chemiluminescence reader, comprising:

1) the chemiluminescent conjugate is provided by the chemiluminescent substance labeling a primary immunoconjugate of the target analyte specific;

2) the capture agent is a non labeled secondary immunoconjugate of the target analyte specific, which is featured with the paired specific binding characteristics of the first immunoconjugate;

3) the solid membrane is pasted on the support material, the capture agent is coated on the solid membrane in a scattered, distributed manner, and the coating area covered by the capture agent on the solid membrane is positively related to the linear detection range of the chemiluminescent immunochromatographic detection, and preferably, the coating amount per unit area of the capture agent molecules on the solid membrane is scatter distributed and do not overlap and aggregate;

4) the chemiluminescent conjugate is mixed with the testing sample to form a sample mixture, the primary immunoconjugate in the sample mixture specifically binds with the target analyte to form the first complex of an analyte-chemiluminescent conjugate, wherein the sample mixture is loaded and flowed forward through the solid membrane and is absorbed in the water absorbent pad, and the target analyte is captured by the secondary immunoconjugate on the solid membrane to form the second complex of a chemiluminescent conjugate-analyte-secondary immunoconjugate, and is immobilized on the solid membrane;

5) the testing buffer is a water-soluble buffer salt solution, and is loaded and made to flow through the solid membrane following the completion of capture and immobilization, and is further absorbed by the water absorbent structure, and cleans up the unbound and unimmobilized label and chemiluminescent conjugate on the solid membrane, and completes the cleaning process of the solid membrane;

6) the water absorbent structure absorbs the water flowing through the solid membrane, and locates at the distal side of the solid membrane and forms a direct connection with the solid membrane, a water absorbent paper pad is preferred;

7) following the completion of the cleanup process of the solid membrane by the testing buffer, the solid membrane is placed for the detection of the amount of light by the chemiluminescence reaction solution and the chemiluminescence reader.

The capture agent is coated on the solid membrane in a scattered and distributed manner and includes three different types of distribution manner, an evenly scattered distributed coating, a gradient scattered distributed coating, and a flaky scattered distributed coating.

The evenly scattered distributed coating is referred to the full covered solid membrane coating by the capturing agent with the same concentration and the same coating amount, forming a uniform distribution of the coated capture agent on the solid membrane. The gradient scattered distributed coating is referred to the increased coating concentration of the capture agent on the solid membrane from proximal side to distal side, forming a gradiently distribution of the coated capture agent on the solid membrane. The flaky scattered distributed coating is referred to the solid membrane divided different sections and coated by the a concentration of capturing agent in certain areas and is completely absent of capture agent in other areas, forming a segmental or flaky or piece by piece scattered distribution on the solid membrane.

The solid membrane refers to a nitrocellulose membrane and the other membranes that are porous and have similar protein binding capacities to a nitrocellulose membrane, comprising nitrocellulose membranes, polyvinylidene fluoride membranes (PVDF), nylon membranes and DEAE cellulose membranes.

The chemiluminescent label is preferred a microparticle structure, including latex microspheres, color microspheres, and magnetic microbeads. Color microspheres include color polymer microspheres and colloidal gold solution.

Both the chemiluminescent conjugate and the capture agent include a immunoconjugate, comprising antibodies, antigens, biotins, avidin and their analogues. For the avidin analogues, straptavidin is the most common choice for this detection.

The chemiluminescent label used for the labeling of the immunoconjugates can either be the direct luminescent labels as acridine ester and acridine sulfonamide, enzymic catalyzed luminescent labels as horseradish peroxidase and alkaline phosphatase, or the electrochemiluminescent label as tripyridine ruthenium.

The most widely used chemiluminescent immunoassays in clinical detection include the direct assay using luminophore label, or indirect assay using enzyme label. In a direct assay, the luminophore labels used are acridinium and ruthenium esters with hydrogen peroxide in an alkaline state substrate, while the enzymatic labels used in indirect methods are alkaline phosphatase with adamantyl 1, 2-dioxetane arylphosphate (AMPPD) substrate and horseradish peroxidase with luminol or its derivatives as substrate.

The chemiluminescence reaction solution includes a direct luminescence reaction solution containing hydrogen peroxide in an alkaline state, an enzymatic luminescence reaction solution in which luminol and its derivatives are luminescent substrates, and an electrochemiluminescence on the electrodes in a ruthenium terpyridine structural labels solution.

The chemiluminescent conjugate is in the form of lyophilized powder.

The solid membrane is provided with a blood cell separation structure with direct connection at the proximal side, wherein the blood cell separation structure includes a blood cell separation membrane pad or a membrane pad treated with the antibody against red blood cells.

The solid membrane comes with a liquid dispersion membrane pad with direct connection at the proximal side, wherein the liquid dispersion membrane pad includes a glass fiber membrane pad or a polyester fiber membrane pad.

The detection structure in this invention can be assembled in three possible combinations: 1) The solid membrane is pasted on the support object and the other components are pasted in this specific order: first the liquid dispersion membrane connects to a blood cell filtering structure and further connects to the proximal side of the solid membrane, then a water absorbent pad downstream and connects to the distal side of the solid membrane; 2) The solid membrane is pasted on the support object and the liquid dispersion membrane is on the upstream and connects to the proximal side of the solid membrane, a water absorbent pad downstream and connects with the distal side of the solid membrane; 3) The solid membrane is pasted on the support object and the blood cell filtering structure is upstream and connects to the proximal side of the solid membrane, a water absorbent pad on the downstream and connects with the distal side of the solid membrane. These assembles can be connected during use and detached when not in use condition.

The detection assay of the testing strip comprises a joint combination of biotin/avidin detection system, therefore the sample mixture includes the chemiluminescent label labeled primary immunoconjugate, a biotin labeled secondary immunoconjugate and the testing sample, and the solid membrane coated with unlabeled avidin and its analogues as the capture agent in the detection.

The chemiluminescent immunochromatographic assay comes with a chemiluminescent reader, which is able to quantitatively detect the amount of light being induce to emit from the solid membrane.

The operation of the membrane based chemiluminescence immunochromatographic detection assay includes the following steps:

1) take the solid membrane structure coated with the capture agent in the scattered, distributed manner, connect the liquid dispersion membrane and/or blood cell separation structure at the proximal side in turn, connect the water absorption structure at the distal side then, and place it at a level position;

2) take the sample, add it into a tube with the chemiluminescent conjugate, take the test solution and add it into the tube again, mix it, and form the sample mixture to be tested;

At this moment, the target analyte in the sample is bound with the chemiluminescent conjugate labeled by the first immunoconjugate (primary antibody) and form the first complex of the analyte+chemiluminescent conjugate, ie the complex of luminous substance+first immunoconjugate (primary antibody)+the target analyte (antigen), and this completes the first testing reaction.

3) add the sample mixture onto the liquid dispersion membrane and allow it to flow forward through the blood cell separationstructure and the solid membrane, and is absorbed in the water absorbent pad;

At this moment, the first complex is captured and immobilized specifically by the capture agent coated on the solid membrane and forms the second complex of the chemiluminescent conjugate+target analyte+capture, ie the complex of luminous substance+first immunoconjugate (primary antibody)+the target analyte (antigen)+second immunoconjugate (secondary antibody), and this completes the second testing reaction.

4) take and add the testing buffer onto the liquid dispersion membrane and allow it to flow forward through the blood cell separation structure and the solid membrane, and is absorbed in the water absorbent pad;

At this moment, the testing buffer cleans up the chemiluminescent substance and the conjugate which are is specifically bound and immobilized on the solid membrane, and this produces a low noise nonspecific detection background, and completes the third testing reaction.

5) take the solid membrane, transfer it to the chemiluminescence reaction solution and the chemiluminescence reader for the quantitative detection of the amount of light from the solid membrane;

6) calculate the concentration of the target analyte in the testing sample based on the standard curve and complete the test.

An application of the chemiluminescence immunochromatographic detection assay in the development of immunoassay reagent products.

The present disclosure has following advantages due to the above technical solutions:

1. This invention utilizes the solid membrane as the carrier of chemiluminescence reaction, and all separation and detection processes of the target analyte are carried out on the solid membrane, so that the separation and operating process of the chemiluminescence detection is only performed by loading liquid sample and testing buffer onto the solid membrane, thereby simplifying the operation and improving the detection throughput of the detection device. The immunochemiluminescence detection includes the separation of the analyte and the quantitative detection after separation. The current tubular chemiluminescence immunoassay requires that all separation and detection processes of the target analyte are carried out on magnetic particles, which need temperature control and complicated mechanical operations and often become the technical barrier to expand detection capacity. This invention is designed so that all separation and detection processes happen on the solid membrane, and all the operation of binding, separation and cleaning are completed by a simple loading step of a liquid and flowing through the solid membrane. It does not require temperature control and reaction incubation or even cold chain transportation, which simplifies the detection process, not only reducing the cost, but also effectively improving the timeliness of clinical detection and the feasibility of popularization and promotion of immunochemiluminescence products.

2. This invention designs the capture agent been coated on the solid membrane in a scattered distributed manner, which give a free and maximized exposure space to the chemiluminescence labeled immunoconjugate, so that the chemiluminescence labeled immunoconjugate can be freely captured and immobilized by the capture agent on the solid membrane and expose to luminescence freely for the luminescence to be detected efficiently. This invention can effectively avoid the luminescence being partially covered and quenched due to the concentrated spray coating of the capture agent on the test line and followed by the concentrated and aggregated binding of chemiluminescent conjugates to the test line in the current technology.

3. With the designation of the capture coating on solid membrane in the scatter distributed manner, the invention not only avoids the influence of the aggregation of chemiluminescent conjugates on the solid membrane during light detection, but also establishes the relationship between the linear detection range and the coating area of the capture agent, which makes it possible for the future development of the particular test project.

4. This invention utilizes the solid membrane as the carrier of the capture agent and has all of the processes of capturing, separating and detecting being completed on the solid membrane. Because the solid membrane used in this invention, such as nitrocellulose membrane, is multiple porous materials, the surface volume of the solid membrane is significantly larger than that of the magnetic particles currently used, so the coating area and further linear detection range is much larger than that of the magnetic particles. It highly increases the detection capacity efficiency compared to the current magnetic particle technology.

5. This invention designs the loading liquid solution onto the test membrane being the only main operation procedure for the detection. It saves the multiple steps from the current tubular magnetic particle procedure as temperature control, incubation, suspension, magnetic separation. Meanwhile, the detection time is faster than the current tubular magnetic particle assay because the liquid migration on the solid membrane is the only time-limited procedure to carry over. The clean up procedure by the testing buffer is also designed to speed up the process, so the time to reach the reaction plateau of testing is very short and very fast.

6. This invention is designed so that lyophilized powder is the form of the chemiluminescent conjugate, which simplifies the storage and transportation process and can be stored at room temperature, which makes it able to avoid the cold chain transportation that is currently used in the chemiluminescent detection reagent.

7. The invention comes with a chemiluminescence reader, which can be used for quantitative detection in various environments, and improves future application.

### Terminology

1. Solid membrane: refers to nitrocellulose membranes and the membrane structural materials that have similar protein binding properties, multiple pores and are water insoluble. Nitrocellulose membranes, polyvinylidene fluoride membranes (PVDF), nylon membranes and DEAE cellulose membranes are commonly used materials on the market.

2. label: also called indicator, chemiluminescent substance, refers to a substance that can produce measurable signal to be used as a marker to directly detect and obtain measurable values in detection technology. This invention uses chemiluminescent substances including horseradish peroxidase and alkaline phosphatase for enzymatic chemiluminescence immunoassay (CLEIA), acridine esters and acridine sulfonamides for direct chemiluminescence immunoassay (CLIA), Tripyridine ruthenium and its derivative N-hydroxysuccinamide (NHS) ester for electrochemiluminescence immunoassay (ECLI).

5. Immunoconjugate: refers to substance that can directly or indirectly form specific binding with the analyte or the substance to be tested, including antigen, antibody, biotin, avidin and its analogues, including primary immunoconjugate and secondary immunoconjugate.

3. Capture agent: refers to the substance that can be coated on the solid membrane and has specific immune binding characteristics of the target analyte and paired bingding feature with the primary immunoconjugate, including antigens, antibodies, biotin, avidin and its anologues, also called secondary immunoconjugate in this invention.

4. Analyte: refers to a specific target test substance, which exists in the sample to be tested, also called target analyte.

6. Scattered distributed coating: refers to the condition that the capture agent molecules are coated on the solid membrane in a dispersed or scattered distribution or manner, and it has no obvious superposition, accumulation, overlap and aggregation between the molecules, which is significantly different from the characteristics of local high concentration aggregation coating formed by the capture agent through spraying or dispensed coating in the existing membrane based immunochromatography detection technology.

7. Evenly scattered distributed coating: the capture agent evenly coated on the solid membrane in a scattered and distributed manner.

8. Gradient scattered distributed coating: the capture agent is gradiently coated on the solid membrane in a way that longitudinal density gradiently increases and horizontal density is evenly distributed.

9. Flaky scattered distributed coating: The capture agent is flaky coated on the solid membrane in a segmental or flaky or piece by piece and scattered distribution. It is partially coverage for the solid membrane rather than coating all solid membranes in a way. It prefers the horizontal density is evenly distributed.

10. Conjugate: the measurable label labeled product, also called detector, in this invention a chemiluminescent substance as the label to labels immunoconjugate to form the chemiluminescent conjugate.

11. Immunochromatography: also known as a lateral flow test, and is a simple and fast assay intended to detect the presence or concentration of an analyte in a liquid sample through a solid membrane based reaction.

### BRIEF DESCRIPTION OF THE DRAWINGS

FIG. 1 is a a schematic diagram of the operation process of this invention;

FIG. 2 is a a schematic diagram of the basic structure of this invention;

FIG. 3 is a a schematic diagram of the basic structure of the evenly scattered distributed coating;

FIG. 4 is a a schematic diagram of the basic structure of the gradient scattered distributed coating;

FIG. 5 is a a schematic diagram of the basic structure of the flaky scattered distributed coating;

FIG. 6 is a a schematic diagram of the detection structure with blood cell separation structure;

FIG. 7 is a a schematic diagram of the integrated structure of this invention.

The marks in the figures are as follows:

Solid membrane 1; capture agent 2; water absorbent structure 3; liquid dispersion membrane 4; support pad 5; evenly scattered distributed coating 6; gradient scattered distributed coating 7; flaky scattered distributed coating 8; blood cell separation structure 9; chemiluminescent conjugate in the form of lyophilized powder 10; chemiluminescent conjugate container 11; testing buffer 12; testing buffer container 13; chemiluminescent reaction solution 14; sample to be tested 15; chemiluminescent conjugate sampling structure 16; testing buffer sampling structure 17; chemiluminescent reader 18; measuring chamber 19

### DETAILED DESCRIPTION OF EMBODIMENTS

This invention will be described in detail below with reference to the accompanying drawings and embodiments, but these accompanying drawings and embodiments are not intended to limit the invention.

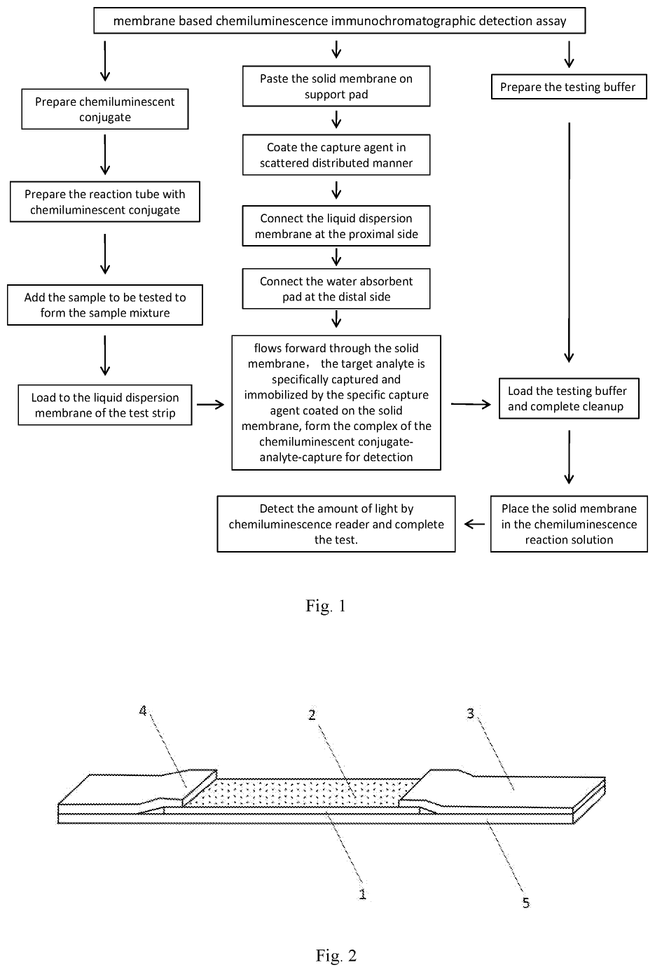

As shown in FIG. 1, the technical procedure of the invention. Take PVC supporting card, paste the solid membrane (nitrocellulose membrane is the common selection) on it; take a capture agent of the secondary antibody or antigen (immunoconjugates) and coat to the solid membrane in scatter distributed manner; take chemiluminescent substances (commonly use three categories as direct luminous indicators of acridine ester compounds and acridine sulfonamide compounds, enzyme chemiluminescences of horseradish peroxidase and alkaline phosphatase, and electrochemiluminescence of tripyridyl ruthenium) and label the primary antibody or antigen to prepare the chemiluminescence conjugates; prepare a testing buffer. While in use, take the coated membrane card, connect a water absorbent pad (commonly water absorption paper pad) at the distal side and connect a liquid dispersion membrane (commonly use glass fiber membrane and polyester fiber membrane) at the proximal end to form a test strip; take testing sample and add into the chemiluminescence conjugates to form the conjugate mixture and start the first testing reaction and form the first complex of the detection analyte-chemiluminescent conjugate; load the conjugate mixture onto the liquid dispersion membrane and have the liquid flow forward through the solid membrane and absorbed in water absorbing pad, and the detection analyte is captured and imobolized on the solid membrane by the secondary immunoconjugate to form the second complex of the detection analyte-chemiluminescent conjugate-secondary immunoconjugate, and the completion of the second testing reaction. Take the testing buffer, load onto the liquid dispersion membrane, flow forward through the solid membrane and water absorption pad, repeat 1 time or more, and the unbound and unimmobilized chemiluminescent substances are cleaned up from the solid membrane. Take the solid membrane, transfer to complete the chemiluminescent measurement with chemiluminescent reaction solution and chemiluminescent reader, and get the test result. The molecule detected by the immunoassay is often referred to as an “analyte”

As shown in FIG. 2, the basic structure of the invention for membrane based chemiluminescence immunochromatography includes the solid membrane 1 pasted on PVC support card 5, the capture agent 2 coated on solid membrane 1 in scatter distributed manner, the water absorbing pad 3 connected to the distal end of the solid membrane 1, and the liquid dispersion membrane 4 connected to the proximal end of the solid membrane. It includes an attached structure and a detached structure. The attached structure is provided with that the structure parts attached together as the order of liquid dispersion membrane 4 connected to the solid membrane 1 and connected to the water absorbing pad 3. The detached structure is provided with the structure parts detached status and assembled when it is in use. It does not provide the indicator conjugate pad as the existing chromatography technology do on the basic structure and the indicator conjugate is provided separately. It does not include the high concentrated test line (T line) on the solid membrane as the existing chromatography technology do as well.

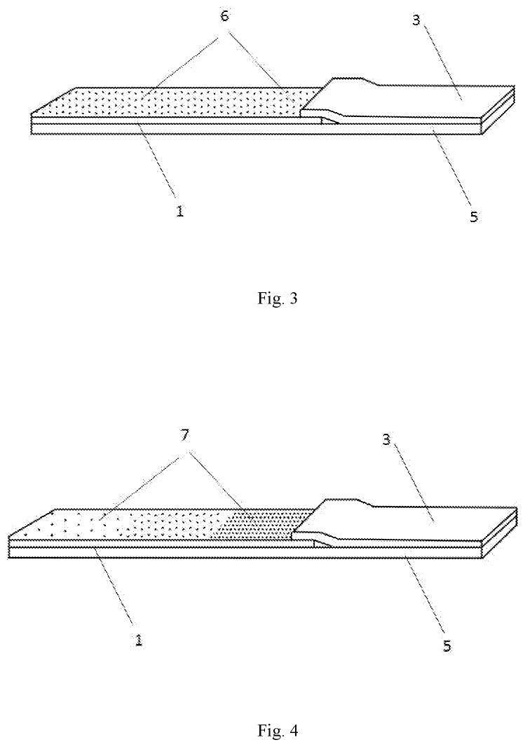

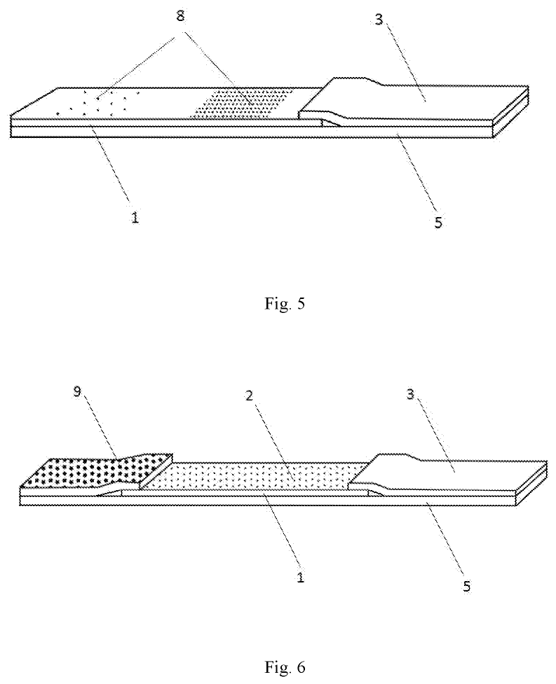

As shown in FIGS. 3, 4, 5, the three ways of the capture agent coating in scatter distributed manner in this invention, FIG. 3 shows the evenly scattered distributed coating 6, the capture agent evenly coated on the solid membrane 1 in scatter distributed manner, FIG. 4 shows the gradient scattered distributed coating 7, the capture agent is gradiently coated on the solid membrane 1 in a way of longitudinal density gradient increase and horizontal density distribution evenly, FIG. 5 shows the flaky scattered distributed coating 8, The capture agent is franctionly coated on the solid membrane in the segmental rather than all solid membranes in a way of the longitudinal density gradient increases and the horizontal density distribution evenly.

As shown in FIGS. 2, 6, the detection structure with blood cell separation of the invention includes the solid membrane 1 pasted on PVC support card 5, the capture agent 2 coated on solid membrane 1 in scatter distributed manner, the water absorbing pad 3 connected to the distal end of the solid membrane 1, and the blood cell separation membrane 9 connected to the proximal end of the solid membrane. It includes an attached structure and a detached structure as well. The attached structure is provided with that the structure parts attached together as the order of blood cell separation membrane 9 connected to the solid membrane 1 and connected to the water absorbing pad 3. The detached structure is provided with the structure parts detached status and assembled when it is in use. It does not provide the indicator conjugate pad as the existing chromatography technology do on the basic structure and the indicator conjugate is provided separately. It does not include the high concentrated test line (T line) on the solid membrane as the existing chromatography technology do. However, the part connecting the proximal end with the blood cell separation structure 9 can be further connected with the liquid dispersion membrane 4.

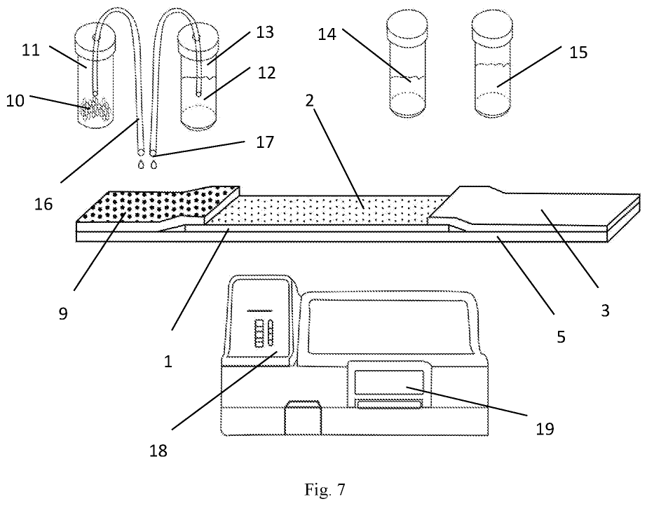

As shown in FIG. 7, the combined detection structure of the invention includes a solid membrane 1, a capture agent 2, a water absorption structure 3, a liquid dispersion membrane 4, a support card 5, a blood cell separation structure 9, a chemiluminescent conjugate 10, a chemiluminescent conjugate container 11, a testing buffer 12, a testing buffer container 13, a chemiluminescent reaction solution 14, a sample 15, a chemiluminescent conjugate sampling structure 16, a testing buffer sampling structure 17 Chemiluminescence detector 18, Chemiluminescence measurement chamber 19, wherein the capture agent 2 is coated on solid membrane 1 in scatter distributed manner, solid membrane 1 is pasted on support card 5, and the two sides of solid membrane 1 are respectively connected with liquid dispersion membrane 4 and/or blood cell separation structure 9 at the proximal side and water absorption structure 3 at the distal side. The chemiluminescent conjugate 10 is kept in the chemiluminescent conjugate container 11, and testing buffer 12 is kept in the testing buffer container 13. The chemiluminescent conjugate sampling structure 16 and the testing buffer sampling structure 17 are provided accordingly. At the same time, it also provides a chemiluminescence reaction solution 14 and a chemiluminescence reader 18. It includes a chemiluminescence reading chamber 19 located inside the chemiluminescence reader 18 for quantitative detection of luminescence. Samples 15 will be provided for testing as well.

The performance of the invention would be carried out by the following steps. Take the solid membrane 1 first and paste it onto the support card 5, prepare the coating solution of low concentration capture agent 2 (secondary antibody or antigen), coat on the solid membrane 1 in scatter distributed manner with the full coverage or segmental coverage of the membrane. Take the coated solid membrane 1 with the capture agent 2, connect with the liquid dispersion membrane 4 and/or blood cell separationstructure 9 at the proximal side of the membrane and the water absorption structure 3 at the distal side of the membrane and form the detection structure of the invention. Take the sample 15 and add into the chemiluminescent conjugate container 11, take the testing buffer 12 and add into the chemiluminescent conjugate container 11, the sample is mixed with chemiluminescent conjugate 10 (primary antibody or antigen) to prepare a testing sample mixture, and then start the first testing reaction and form the first complex of the detection analyte-chemiluminescent conjugate. Then, the testing sample mixture is transferred onto the liquid dispersion membrane 4, and flows forward through the blood cell separation structure 9, the solid membrane 1, and is absorbed in the water absorbing pad 3. During this process, the first complex is specifically captured and immobolized by the specific capture agent (secondary antibody or antigen) on the solid membrane 1, form the second complex of the chemiluminescent conjugate-analyte-capture, ie the complex of chemiluminescent substance—the first immunoconjugate-testing analyte—the second immunoconjugate, complete the second testing reaction. Take the testing buffer 12, load onto the liquid dispersion membrane 4, and flows forward through the blood cell separation structure 9, the solid membrane 1, and is absorbed in the water absorbing pad 3. During this process, the testing buffer 12 clean up the chemiluminescent substances and the conjugates which are not specifically bound and immobilized on the solid membrane 1, create a low level nonspecific detection background, and completes the third testing reaction. Take the solid membrane 1, transfer to complete the chemiluminescent measurement with chemiluminescent reaction solution and chemiluminescent reader, and get the test result.

The following experiments further describe the technical effects of the present disclosure with reference to specific experimental examples, are not intended to limit the invention. Unless otherwise specified, the experimental methods used in the following experiments are conventional assays, the used materials and reagents are commercially available.

Experiment 1: Comparison with Conventional Enzyme Chemiluminescence Assay in this Invention:

I. Preparation of Enzyme Chemiluminescence Conjugates:

Preparation of HRP labeled anti-human myoglobin monoclonal antibody solution by using conventional horseradish peroxidase (HRP) labeling method of sodium periodate oxidation assay. At first oxidized sugar molecules on the HRP surface to aldehyde groups, and then coupled it with amino groups on the monoclonal antibody to form HRP labeled anti-human myoglobin monoclonal antibody. Specifically, weighed 5 mg HRP and dissolved it in 1 ml purified water, added 0.2 ml of the newly prepared 0.1M NaIO4 solution and stirred for 20 minutes at room temperature in dark, and then put the above solution into a dialysis bag, dialyzed the above solutions with 1 mM PH4.4 sodium acetate buffer solution, stand for one night at 4° C., added 20 μL 0.2M PH9.5 carbonate buffer solution to increase the PH of the above aldehyde extended HRP to 9.0-9.5, and then immediately added 10 mg anti-human myoglobin primary monoclonal antibody in 1 ml 0.01M carbonate buffer solution, and gently stirred it at room temperature for 2 hours, added 0.1 ml of newly prepared 4 mg/ml NaBH4 solution, mixed them evenly, and then stand at 4° C. for 2 hours, poured the solution into a dialysis bag, dialyzed against 0.15M PH7.4 phosphate buffer solution at 4° C. for one night, and purified it through chromatographic column after dialysis. Diluted the above solution with 0.15M PH7.4 phosphate buffer to 10 million light-emitting units (RLUs)/ul, 5 ul/tube each before use as enzyme chemiluminescence conjugate tube.

II. Preparation of Coated Solid Membrane:

Took the paired anti-human myoglobin secondary monoclonal antibody as the capture agent, diluted with 50 mM phosphate buffer pH7.4 to 1.0 mg/ml as control coating buffer, diluted with 50 mM phosphate buffer containing 0.1 mg/ml mouse IgG pH7.4 to 250 ug/ml as the first coating buffer, 62.5 ug/ml as the second coating buffer and 15.6 ug/ml as the third coating buffer.

Control coating: turned on the membrane dispenser, loaded 1.0 mg/ml control coating buffer, took a 5 mm wide nitrocellulose membrane PVC card, and started coating. Coating setting: movement speed 10 mm/s, and liquid dispensing speed 1.5 μl/cm. Put the coated membrane card at 37° C. for 6 hours, and then stored it in a drying container for use.

Evenly scattered distributed coating in this invention: Took 250 ug/ml first coating buffer and soaked to coat on the full 5 mm wide nitrocellulose membrane evenly, and put the coated membrane into a 37° C. for 6 hours, and then put it into a drying container for use.

Gradient scattered distributed coating in this invention: Took a 5 mm wide nitrocellulose membrane PVC card and marked it as the upper, middle and lower sections, and then sprayed to coat respectively as 250 ug/ml first coating buffer for upper section, 62.5 ug/ml second coating buffer for middle section and 15.6 ug/ml third coating buffer for lower section. Coating setting:: movement speed 10 mm/s, and liquid dispensing speed 1.5 μl/cm. Put the coated membrane card at 37° C. for 6 hours, and then stored it in a drying container for use.

Flaky scattered distributed coating in this invention: Took a 5 mm wide nitrocellulose membrane PVC card and marked it as the upper, middle and lower sections, and then sprayed to coat 250 ug/ml first coating buffer for upper section and 15.6 ug/ml third coating buffer for lower section, and leave the middle section as a blank. Coating setting: movement speed 10 mm/s, and liquid dispensing speed 2.0 μl/cm. Put the coated membrane card at 37° C. for 6 hours, and then stored it in a drying container for use.

III. Test Product Assembly:

Turned on the dehumidifier to reduce the humidity in the operating room to less than 25%. Pasted absorbent paper pad and glass fiber liquid dispersion membrane on two side of the coated solid membrane. Placed the assembled card on the cutter and cut it into 4.0 mm strips. Put the strips into the aluminum foil bag and sealed for use as the coated control strips, the coated scatter distributed evenly strips, the coated scatter distributed gradiently strips, and the coated scatter distributed Flaky strips respectively. Prepared the testing buffer of 30 mM Tris pH8.5, 1% NP40, 1M sodium chloride and 0.5% sodium casein in H2O.

IV. Experimental Materials:

The following materials were used in this experiment: 2 mL test tube as chemiluminescence conjugate container and testing buffer container, CN140 nitrocellulose membrane from Sartorius as solid membrane, and SB08 glass fiber membrane from Shanghai Gold Standard Company as liquid dispersion membrane, ISOF Flow Dispenser from Imagen Technology as membrane dispenser, CE Strip Cutter from HanGan China as cutter, Glomax Multi Jr Reader from Promega as chemiluminescence reader, West Femto Peroxide Solution from Thermo Scientific as a enzyme chemiluminescence reaction solution.

V. Experimental Methods

Preparation of myoglobin solution: Took human myoglobin solution with known concentration and diluted it with sample dilution buffer (1% BSA, 100 mM glycine, 50 mM PBS, 150 mM NaCl, pH7.4) to prepared a series of myoglobin solutions with concentrations of 3, 30, 100, 300, 1000, 3000 ng/ml.

Research group: added the prepared myoglobin solutions 50u1/tube into different enzyme chemiluminescence conjugate tubes respectively to prepared a conjugate mixture. Took the above prepared the coated scatter distributed evenly strips, the coated scatter distributed gradiently strips, and the coated scatter distributed Flaky strips, added 50 ul prepared conjugate mixture to the liquid dispersion membrane, stand for 2 minutes, added 25 ul testing buffer onto the liquid dispersion membrane, stand for 1 minute, and then added 25 ul testing buffer and stand for 5 minutes, remove the liquid dispersion membrane and water absorbing pad from the strip, Placed the nitrocellulose membrane in the test tube containing enzyme chemiluminescence reaction solution, read the luminescence RLUs on the chemiluminescence reader in triplicate, and calculate the average.

Control group: Performed the test with the above method in research group using the coated control strips and calculate the results.

VI. Experimental Result

The solid membrane was used as the carrier of chemiluminescence reaction in this invention, and the experiments were carried out by using the coating of scatter distributed evenly, scatter distributed gradiently, and the scatter distributed flaky comparing with the control of the conventional coating. The results were shown in Table 1. The test results of the three coated products in the research group showed a good concentration luminescence correlation. When the upper test limit was set to 3000 ng/ml and the linear detection range was 3-3000 ng/ml, the correlation coefficient r2 of evenly distributed coating was 0.977, gradient dispersion distribution coating was 0.968, sectional dispersed distribution coating was 0.985, and showed good linear response; But in the control test group, the conventional coating strip only showed a small concentration—luminescence reaction relationship, its linear range was far smaller than that of this invention groups. Only the concentration between 3 ng/ml and 30 ng/ml was a linear response when we choose the test range from 3-3000 ng/ml, which does not meet the clinical test requirements. It shows that the technology in this invention is superior to the existing technology and suitable for enzyme chemiluminescence detection.

TABLE 1
Comparison of This Invention with Conventional
Enzyme Chemiluminescence Assay

| | Concentration (ng/ml)/(1000 RLUs) | | | | | | |
|---|---|---|---|---|---|---|---|
| Group | 3 | 30 | 100 | 300 | 1000 | 3000 | r² |
| Evenly | 593 | 1821 | 4849 | 8068 | 10487 | 15117 | 0.977 |
| coated | | | | | | | |
| Gradiently | 829 | 2576 | 5479 | 11751 | 13463 | 16045 | 0.968 |
| coated | | | | | | | |
| Flaky | 548 | 2048 | 6036 | 9080 | 12396 | 13845 | 0.985 |
| coated | | | | | | | |
| Control | 1282 | 3682 | 4214 | 4307 | 4243 | 4237 | 0.554 |

Experiment 2: Comparison of this Invention with Conventional Direct Chemiluminescence Assay:

I. Preparation of Direct Chemiluminescence Conjugate:

Use acridine ester (NSP-SA-NHS) as a direct chemiluminescence substance to react with monoclonal antibody containing amino groups. In alkaline conditions, acridine ester NHS reacts with monoclonal antibody to form stable amide bonds, and labels anti-human myoglobin primary monoclonal antibody with acridine ester. Specifically, prepared 2.5 mg/mL acridinium-DMSO stock solution and prepared 0.5 mg/mL antibody reaction solution using 0.2 M NaHCO3 (pH=9.0). Took 10 μL diluted acridine ester stock solution (2.5 mg/mL), Diluted with 90 μL anhydrous DMSO by 10-fold to prepared cridine ester working solution (0.25 mg/mL). Diluted 50 μg of antibody to 300 μL with 0.2 M NaHCO3 (pH 9.0), and added 10 μL of acridine ester working solution (0.25 mg/mL). Stand for 1 h at room temperature in dark. Seal, added 100 μL of labeling stop buffer (10% lysine, 0.2 MNaHCO3, pH 9.0) and mixed for 30 minutes at room temperature. Dialysis against 10 mM PB pH 6.5 buffer for overnight at 4° C., then purified with molecular sieve. Diluted with 0.15M PH7.4 phosphate buffer to 10 RLUs/ul, 5 ul/tube each before use as direct chemiluminescence conjugate tube.

II. Preparation of Coated Solid Membrane:

Same as “control coating” and “Scatter distributed coating evenly in this invention” in “Experiment 1”.

III. Test Product Assembly:

Same as “Experiment 1”.

IV. Experimental Materials:

Same as “Experiment 1”.

V. Experimental Methods

Preparation of myoglobin solution: same as “Experiment 1”.

Research group: Perform the test with the method and “the coated scatter distributed evenly strips” in “Experiment 1” using direct chemiluminescence conjugate in this experiment. After the completion of reaction, remove the liquid dispersion membrane and water absorbing pad from the strip, Placed the nitrocellulose membrane in a detection tube. Took a thin tubing, Placed one side in the detection tube and leave another side outside and connect the outside end to a syringe with acridine ester chemiluminescence reaction solution, and then put the detection tube in the luminescence reader. Started automatically counting and push to added acridine ester chemiluminescence reaction solution immediately, record luminescence RLUs for 5 s, repeat the test for 3 times, and calculate the average.

Control group: Perform the test with the method and “the coated control strips” in “Experiment 1” using direct chemiluminescence conjugate and the “research group” method in this experiment, and calculate the average.

VI. Experimental Result

The solid membrane was used as the carrier of chemiluminescence reaction in this invention, and the experiments were carried out by using the coating of scatter distributed evenly and direct chemiluminescence conjugate comparing with the control of the conventional coating. The results were shown in Table 2. The test results of the coated products in the research group show a good concentration luminescence correlation. When the upper test limit was set to 3000 ng/ml and the linear test range was 3-3000 ng/ml, the correlation coefficient r2 of evenly distributed coating was 0.962. But in the control test group, the conventional coating strip only showed a small concentration—luminescence reaction relationship, its linear range was far smaller than that of this invention groups. Only the concentration between 3 ng/ml and 30 ng/ml was a linear response, which does not meet the clinical test requirements. It shows that the technology in this invention is superior to the existing technology and suitable for enzyme chemiluminescence detection.

TABLE 2
Comparison of this invention with conventional
direct chemiluminescence assay

| | Concentration (ng/ml)/(1000 RLUs) | | | | | | |
|---|---|---|---|---|---|---|---|
| Group | 3 | 30 | 100 | 300 | 1000 | 3000 | r² |
| Research | 1023 | 3187 | 8976 | 15432 | 18793 | 19987 | 0.962 |
| group | | | | | | | |
| Control | 853 | 3033 | 3208 | 3524 | 3367 | 3988 | 0.554 |
| group | | | | | | | |

Experiment 3: Comparison of this Invention with Conventional Latex Conjugate Assay

I. Preparation of Enzyme Chemiluminescence Conjugate Using Latex Particles:

HRP-antibody-latex conjugates were prepared by labeling the horseradish peroxidase (HRP) labeled anti-human myoglobin monoclonal antibody prepared by “Experiment 1” using conventional latex particle antibody labeling method. Specifically, Took a 2.0 mL test tube, added primary washing solution (10 mM IVIES PH 5.5, T20 0.05%) 1 ml, added 12.5 μL of size 300 nm latex particle stock solution from Du Biological Company, mixed well, and centrifuge at 10,000 rpm for 20 min; prepared 50 mg/mL EDC and NHS solution with the primary washing solution, remove the supernatant after centrifugation, added 750 μL of primary washing solution, ultrasonic, added 100 μL EDC solution and 150 μL NHS solution, mixed well, activate at 37° C. for 15 min, centrifuge at 10,000 rpm for 20 min; remove the supernatant after centrifugation, added 1 mL coupling buffer (10 mM MES PH 5.0, PC300 0.04%). After ultrasonic mixing, centrifuge at 10,000 rpm for 20 min; remove the supernatant after centrifugation, added 1 mL of coupling solution, ultrasonic mixing, added 50 ug HRP-labeled anti-human myoglobin primary monoclonal antibody, conjugate at 37° C. for 2 h, sonicate for 2 min, then centrifuge at 10,000 rpm for 20 min; after centrifugation, remove the supernatant, added 1 mL of blocking solution (10 mM Tris PH8.5, glycine 20 mM, T20 0.050%), ultrasonic mixing, block at 37° C. for 30 min, centrifuge at 10,000 rpm for 20 min; remove the supernatant after centrifugation, added 1 mL of final washing solution (10 mM Tris PH8.5, BSA 0.20%, T20 0.05%), ultrasonic mixing, and centrifuge at 10,000 rpm for 20 min; remove the supernatant after centrifugation, added 1 mL of resuspend solution (10 mM Tris pH 8.5, 0.4% casein sodium, 0.02% Tween 20, 10% trehalose aqueous solution), ultrasonic mixing, and stored at 4° C. for future use. Diluted to 10 RLUs/ul with resuspend solution before use, and 5 ul/tube each before use as latex chemiluminescence conjugate tube.

II. Preparation of Coated Solid Membrane:

Same as “control coating” and “Scatter distributed coating evenly in this invention” in “Experiment 1”.

III. Test Product Assembly:

Same as “Experiment 1”.

IV. Experimental Materials:

Same as “Experiment 1”.

V. Experimental Methods

Preparation of myoglobin solution: same as “Experiment 1”.

Research group: Perform the test with the method and “the coated scatter distributed evenly strips” in “Experiment 1” using latex chemiluminescence conjugate in this experiment, and calculate the average.

Control group: Perform the test with the method and “the coated control strips” in “Experiment 1” using latex chemiluminescence conjugate in this experiment, and calculate the average.

VI. Experimental Result

The solid membrane was used as the carrier of chemiluminescence reaction in this invention, and the experiments were carried out by using the coating of scatter distributed evenly and latex chemiluminescence conjugate comparing with the control of the conventional coating. The results were shown in Table 3. The test results of the coated products in the research group show a good concentration luminescence correlation. When the upper test limit was set to 3000 ng/ml and the linear test range was 3-3000 ng/ml, the correlation coefficient r2 of evenly distributed coating was 0.987. But in the control test group, the coated control strips showed a small concentration—luminescence reaction relationship, its linear range was far smaller than that of this invention groups. Only the concentration between 3 ng/ml and 30 ng/ml was a linear response, which does not meet the clinical test requirements. It shows that the technology in this invention was superior to the existing technology and suitable for latex chemiluminescence detection.

TABLE 3
Comparison of this invention with
conventional latex conjugate assay

| | Concentration (ng/ml)/(1000 RLUs) | | | | | | |
|---|---|---|---|---|---|---|---|
| Group | 3 | 30 | 100 | 300 | 1000 | 3000 | r² |
| Research | 1279 | 3986 | 9654 | 13520 | 19864 | 22211 | 0.9877 |
| group | | | | | | | |
| Control | 562 | 2209 | 2135 | 2378 | 2402 | 2529 | 0.5995 |
| group | | | | | | | |

Experiment 4: Comparison of this Invention with Conventional Colloidal Gold Conjugate Assay

I. Preparation of Colloidal Gold Chemiluminescence Conjugates:

HRP-antibody-colloidal gold conjugates were prepared by labeling the horseradish peroxidase (HRP) labeled anti-human myoglobin monoclonal antibody prepared by “Experiment 1” using conventional colloidal gold antibody labeling assay. Specifically, Took 1.5 mL centrifuge tube, added 1 mL colloidal gold solution with particle size of 50 nm, added 3.6 ul 0.1M potassium carbonate, stirred evenly, added 5 ug HRP-labeled anti-human myoglobin primary monoclonal antibody, added 10 ul/mL 20% BSA after reaction for 10 min, centrifuge at 10000 r/min for 15 min, remove the supernatant, suspend the pellet with 1 mL colloidal gold resuspend solution (30 mM Tris, 0.4% casein sodium, 3% trehalose, 3% sucrose, 0.25% BSA), centrifuge at 10000 r/min for 15 min, remove the supernatant, suspend the pellet with colloidal gold resuspend solution again, with a final volume of 0.5 mL. Diluted to 10 RLUs/ul with colloidal gold resuspend solution before use, 5 ul/tube each before use as colloidal gold chemiluminescence conjugate tube.

II. Preparation of Coated Solid Membrane:

III. Same as “control coating” and “Scatter distributed coating evenly in this invention” in “Experiment 1”.

IV. Test Product Assembly:

Same as “Experiment 1”.

V. Experimental Materials:

Same as “Experiment 1”.

VI. Experimental Methods

Preparation of myoglobin solution: same as “Experiment 1”.

Research group: Perform the test with the method and “the coated scatter distributed evenly strips” in “Experiment 1” using colloidal gold chemiluminescence conjugate in this experiment, and calculate the average.

Control group: Perform the test with the method and “the coated control strips” in “Experiment 1” using colloidal gold chemiluminescence conjugate in this experiment, and calculate the average.

VII. Experimental Result

The solid membrane was used as the carrier of chemiluminescence reaction in this invention, and the experiments were carried out by using the coating of scatter distributed evenly and colloidal gold chemiluminescence conjugate comparing with the control of the conventional coating. The results were shown in Table 4. The test results of the coated products in the research group show a good concentration luminescence correlation. When the upper test limit was set to 3000 ng/ml and the linear test range was 3-3000 ng/ml, the correlation coefficient r2 of evenly distributed coating was 0.987. But in the control test group, the coated control strips showed a small concentration—luminescence reaction relationship, its linear range was far smaller than that of this invention groups. Only the concentration between 3 ng/ml and 30 ng/ml was a linear response, which does not meet the clinical test requirements. It shows that the technology in this invention was superior to the existing technology and suitable for colloidal gold chemiluminescence detection.

TABLE 4
Comparison of this invention with conventional
colloidal gold labeling assay

| | Concentration (ng/ml)/(1000 RLUs) | | | | | | |
|---|---|---|---|---|---|---|---|
| Group | 3 | 30 | 100 | 300 | 1000 | 3000 | r² |
| Research | 971 | 2792 | 6823 | 11851 | 13541 | 19390 | 0.9794 |
| group | | | | | | | |
| Control | 461 | 2304 | 2987 | 2876 | 3102 | 2766 | 0.5486 |
| group | | | | | | | |

Experiment 5: The Comparison Studies Between the Coating Area of the Capture Agent and the Detection Range of the Linear Interval in this Invention.

I. Preparation of Colloidal Gold Chemiluminescence Conjugates:

Same as “Experiment 4”.

II. Preparation of Coated Solid Membrane:

Took the anti-human myoglobin secondary monoclonal antibody as the capture agent, prepared the coating solution at concentrations of 1.0 mg/mL, 0.5 mg/mL, 250 ug/ml, 125 ug/mL and 62.5 ug/mL with 50 mM phosphate buffer pH 7.4 containing 0.1 mg/mL mouse IgG. Past nitrocellulose membrane with width of 10 mm on PVC card and use 1 card per group. Turned on the membrane dispenser and started coating. Group 1, loaded 1.0 mg/mL coating solution and sprayed 1 capture line on nitrocellulose membrane. Group 2, loaded 0.5 mg/mL coating solution and sprayed 2 capture lines on nitrocellulose membrane without overlapping. Group 3, loaded 250 ug/mL coating solution and sprayed 4 capture lines on nitrocellulose membrane without overlapping. Group 4, loaded 125 ug/mL coating solution and sprayed 8 capture lines on nitrocellulose membrane without overlapping. Group 5, loaded 62.5 ug/mL coating solution and sprayed 16 capture lines on nitrocellulose membrane without overlapping. Coating setting: movement speed 10 mm/s, and liquid dispensing speed 1.5 μl/cm. Put the coated membrane card at 37° C. for 6 hours, and then stored it in a drying container containing desiccant for use.

III. Test Product Assembly:

Same as in “Experiment 1” using the coated membrane in this experiment.

IV. Experimental Materials:

Same as “Experiment 1”.

V. Experimental Methods

Preparation of myoglobin solution: same as “Experiment 1”.

Research group: Perform the test with the method in “Experiment 1” using the coated test strips in this experiment, and calculate the average.

VI. Experimental Result

The same amount of capture agent but different coating areas on each test strip was used in this experiment for the analysis of the coating area of the capture agent and the detection range of the linear interval in this experiment. The results were shown in Table 5. The linear detection interval of myoglobin detection increased with the increase of coating area. If the correlation coefficient r2 over 0.95 was considered as linear detection range, the linear range for 1 coating line of 1.0 mg/mL coating solution was 3-100 ng/mL (r2=0.952); the linear range for 2 coating line of 0.5 mg/mL coating solution was 3-300 ng/mL (r2=0.967); the linear range for 4 coating line of 250 ug/mL coating solution was 3-3000 ng/mL (r2=0.958); the linear range for 8 coating line of 125 ug/mL coating solution was 3-3000 ng/mL (r2=0.964); the linear range for 16 coating line of 62.5 ug/mL coating solution was 3-3000 ng/mL (r2=0.969). It suggested that the linear detection interval of chemiluminescence detection in this invention was correlated with the coating area of capture agent and increased by increasing the coating area of capture agent on the solid membrane.

TABLE 5
Comparison between capture agent coating area
and linear interval in this experiment

| | Concentration (ng/ml)/(1000 RLUs) | | | | | | |
|---|---|---|---|---|---|---|---|
| Group | 3 | 30 | 100 | 300 | 1000 | 3000 | r² |
| 1 | 652 | 3102 | 3876 | 3901 | 4122 | 3984 | 0.6270 |
| 2 | 892 | 2810 | 8788 | 9723 | 9251 | 9537 | 0.7495 |
| 3 | 562 | 2902 | 10234 | 15213 | 17572 | 19653 | 0.9586 |
| 4 | 717 | 2919 | 9290 | 15193 | 18477 | 19883 | 0.9642 |
| 5 | 512 | 2124 | 10096 | 15008 | 17529 | 21189 | 0.9687 |

Experiment 6: Comparison Experiment of the Coating Concentration of the Capture Agent with the Linearity Of Detection

I. Preparation of Enzyme Chemiluminescence Conjugates:

Same as “Experiment 4”

II. Preparation of Coated Solid Membrane:

Took the anti-human myoglobin secondary monoclonal antibody with paired binding properties to the anti-human myoglobin first monoclonal antibody as a capture agent, Diluted it with 50 mM phosphate buffer pH 7.4 to prepared the coating solutions at a concentration of 2.0 mg/mL, 1.0 mg/mL, 0.5 mg/mL, 250 μg/mL, 125 μg/mL, 62.5 μg/mL, 31.3 μg/mL and 15.6 μg/mL, respectively. Took the nitrocellulose membrane card with a width of 5 mm, cut it to 2 mm×5 mm strip, and coat the membrane by sinking it into the coating solutions prepared above and drying up with absorbent paper, respectively. Put the coated membrane into a drying oven at 37° C. for 6 hours, and then stored it in a drying container containing a desiccant for later use.

III. Test Product Assembly:

Same as “Experiment 1” using the coated strips in this experiment.

IV. Experimental Materials:

Same as “Experiment 1”.

V. Experimental Methods

Preparation of myoglobin solution: Diluted a known concentration of human myoglobin solution with sample dilution buffer (1% BSA, 100 mM glycine, 50 mM PBS, 150 mM NaCl, pH 7.4) to prepared 3000 ng/mL of myoglobin solution.

Research group: Same as “Experiment 1”, using the colloidal gold enzyme chemiluminescence conjugate and the “coated solid membrane” reagent strips prepared in this experiment, with the concentration of 3000 ng/ml of myoglobin solution for different tests.

VI. Experimental Result

In this invention, the solid membrane was used as the carrier for chemical reaction, and the capture agent was evenly dispersed and distributed to coat the coating solutions of different concentrations for the experiment. The results were shown in Table 6. In this experiment, the same amount of enzyme chemiluminescence conjugate was used with the strips of different coating concentrations of capture agent. When the coating concentration of capture agent was from high to low, the luminescence detection value showed low first, then high, and then gradually declined and showed that its luminescence decreased with the increase of the coating concentration in the high concentration range, but it was a correlated response in the medium and low concentration range. A clear band of aggregated red colloidal gold particles can be observed at the proximal end of the nitrocellulose membrane in 2.0 mg/ml high concentration coating solution and showed low luminescence detection value. This indicated that when the coating concentration of the capture agent was high, the capture agent on the solid membrane was in aggregation and cause the aggregated and overlying of the chemiluminescence conjugate on the membrane, which reduced the effective luminescence detection area, thereby reduce the luminescence detection value. However, when the coating concentration of the capture agent was in medium and low level, the capture agent on the solid membrane was scatter distributed, and it would not cause aggregation formation and maintain a normal effective luminescence detection. This indicated that the luminescence detection value is directly correlated with the size of coating area and the density of capture coating and further bind to the chemiluminescence conjugate, and then there was a positive correlation between the luminescence amount and the coating concentration of the capture agent.

TABLE 6
Comparison experiment of the coating concentration of
the capture agent with the linearity of detection

| | Capture agent coating concentration (μg/mL)/(1000 RLUs) | | | | | | | |
|---|---|---|---|---|---|---|---|---|
| Conjugate | 2000 | 1000 | 500 | 250 | 125 | 62.5 | 31.3 | 15.6 |
| 5.0 uL | 5672 | 11528 | 10440 | 19356 | 11192 | 4848 | 2244 | 716 |

Experiment 7: Comparison Experiment of Biotin/Avidin System in this Invention:

I. Preparation of Enzyme Chemiluminescence Conjugates:

Same as “Experiment 4”.

II. Preparation of Biotyzation Conjugate:

Diluted the anti-human myoglobin second monoclonal antibody to 1.0 mg/ml with 10 mM PBS pH7.4, Took activated biotin (Sigma) in a test tube, then dissolve it with 10 mM PBS pH7.4 to the final concentration of 20 mM, added 13.3 μL of 20 mM activated biotin per 2 mg of anti-human myoglobin secondary monoclonal antibody, mixed well and stand at room temperature for 60 min; after the reaction, dialyzed against 10 mM PBS PH7.4 for overnight, and collected for further use.

III. Preparation of Coated Solid Membrane:

Prepared 100 μg/mL streptavidin (Sigma) with 50 mM phosphate buffer pH 7.4 as control coating solution, and prepared 20 μg/mL streptavidin with 50 mM phosphate buffer pH 7.4 as research coating solution.

Control coating: turned on the membrane dispenser, loaded 100 μg/mL control coating solution, Took a 5 mm wide nitrocellulose membrane PVC card, and started coating. Coating setting:: movement speed 10 mm/s, and liquid dispensing speed 1.5 μl/cm. Put the coated membrane card at 37° C. for 6 hours, and then stored it in a drying container containing desiccant for use.

Research group: use scatter distributed coating evenly in this invention, take 20 ug/ml research coating solution and 5 mm wide nitrocellulose membrane, and soaked the membrane in the coating solution completely for full evenly coating, and put the coated membrane into a 37° C. for 6 hours, and then put it into a drying container containing desiccant for further use.

IV. Test Product Assembly:

Same as “Experiment 1” using the coated strips in this experiment as the coated streptavidin strips.

V. Experimental Materials:

Same as “Experiment 1”.

VI. Experimental Methods

Preparation of myoglobin solution: same as “Experiment 1”.

Took the chemiluminescence conjugates and biotin conjugates (7:3 v/v) prepared in this experiment and mixed to prepared conjugate mixture tubes. The myoglobin solution with different concentrations was added to different tubes of the above-prepared conjugate mixture at 50 μL/tube to form myoglobin conjugate mixture.

Research group: Took the above prepared the evenly coated streptavidin strips, added 50 ul of different concentrations of myoglobin conjugate mixture to the liquid dispersion membrane, stand for 2 minutes, then added 25 ul of test solution to the liquid dispersion membrane, stand for 1 minute and then added 25 ul again, stand for 5 minutes, remove the liquid dispersion membrane and absorbent pad from the test strips, Placed the nitrocellulose membrane into the detection tube with enzyme chemiluminescence reaction solution, read RLUs on the chemiluminescence reader in triplicate, and calculate the average.

Control group: Took the above prepared the control coated streptavidin strips, and test using the research group method in this experiment and calculate the average.

VII. Experimental Result

The solid membrane was used as the carrier of chemiluminescence reaction in this invention, and the experiments were carried out by using the coating of scatter distributed evenly, colloidal gold chemiluminescence conjugate and biotin/avidin system comparing with the control of the conventional coating. The results were shown in Table 7. In this invention, the colloidal gold labeled enzyme chemiluminescence conjugates in biotin/avidin system showed a good concentration-luminescence correlation for myoglobin detection, and when the upper limit of detection was set at 3000 ng/mL, the detection range was 3-3000 ng/mL, and correlation coefficient r² was 0.978. The control detection group with conventional coated strips showed a similar concentration-luminescence response relation, but its linear range was much lower than that of the research group, with a linear response range from 3 and 30 ng/mL only. This indicated that this invention with biotin/avidin systems is better than the existing techniques and works for chemiluminescence detection as well.

TABLE 7
Comparative experiment of biotin/avidin system

| | Concentration (ng/ml)/(1000 RLUs) | | | | | | |
|---|---|---|---|---|---|---|---|
| Group | 3 | 30 | 100 | 300 | 1000 | 3000 | r² |
| Research | 2418 | 6617 | 13226 | 17067 | 21510 | 23292 | 0.9781 |
| group | | | | | | | |
| Control | 1123 | 3201 | 3544 | 3671 | 3459 | 3527 | 0.5019 |
| group | | | | | | | |

These embodiments are merely illustrative of the invention, and various changes can be made to the invention with respect to the structure and connection of the components. Equivalent changes and modifications made based on the content of the invention should fall within the scope of the invention.

What is claimed is:

1. A membrane based chemiluminescence immunochromatographic detection assay, comprising a solid membrane, a capture agent, a chemiluminescence label, a chemiluminescence conjugate, a testing buffer, a chemiluminescence reaction solution, and a chemiluminescence reader, wherein the capture agent is coated on the solid membrane in a scattered, distributed manner, and the coating area covered by the capture agent on the solid membrane is positively related to the linear detection range of the chemiluminescent immunochromatographic detection.

2. The membrane based chemiluminescence immunochromatographic detection assay, comprising the solid membrane, the capture agent, the chemiluminescence label, the chemiluminescence conjugate, the testing buffer, the chemiluminescence reaction solution, and the chemiluminescence reader, comprising:
1) the chemiluminescent conjugate is provided by the chemiluminescent substance labeling a primary immunoconjugate of the target analyte specific;
2) the capture agent is a non labeled secondary immunoconjugate of the target analyte specific, which is featured with the paired specific binding characteristics of the first immunoconjugate;
3) the solid membrane is pasted on the support material, the capture agent is coated on the solid membrane in a scattered, distributed manner, and the coating area covered by the capture agent on the solid membrane is positively related to the linear detection range of the chemiluminescent immunochromatographic detection, and preferably, the coating amount per unit area of the capture agent molecules on the solid membrane is scatter distributed and do not overlap and aggregate;
4) the chemiluminescent conjugate is mixed with the testing sample to form a sample mixture, the primary immunoconjugate in the sample mixture specifically binds with the target analyte to form the first complex of an analyte-chemiluminescent conjugate, wherein the sample mixture is loaded and flowed forward through the solid membrane and is absorbed in the water absorbent pad, and the target analyte is captured by the secondary immunoconjugate on the solid membrane to form the second complex of a chemiluminescent conjugate-analyte-secondary immunoconjugate, and is immobilized on the solid membrane;
5) the testing buffer is a water-soluble buffer salt solution, and is loaded and made to flow through the solid membrane following the completion of capture and immobilization, and is further absorbed by the water absorbent structure, and cleans up the unbound and unimmobilized label and chemiluminescent conjugate on the solid membrane, and completes the cleaning process of the solid membrane;
6) the water absorbent structure absorbs the water flowing through the solid membrane, and locates at the distal side of the solid membrane and forms a direct connection with the solid membrane, a water absorbent paper pad is preferred;
7) following the completion of the cleanup process of the solid membrane by the testing buffer, the solid membrane is placed for the detection of the amount of light by the chemiluminescence reaction solution and the chemiluminescence reader.

3. The detection assay of claim 1, wherein the capture agent is coated on the solid membrane in a scattered and distributed manner and includes three different types of distribution manner, an evenly scattered distributed coating, a Gradient scattered distributed coating, and a flaky scattered distributed coating.

4. The detection assay of claim 1, wherein the solid membrane refers to a nitrocellulose membrane and the other membranes that are porous and have similar protein binding capacities to a nitrocellulose membrane, comprising nitrocellulose membranes, polyvinylidene fluoride membranes (PVDF), nylon membranes and DEAE cellulose membranes.

5. The detection assay of claim 1, wherein the chemiluminescent label is preferred a microparticle structure, including latex microspheres, color microspheres, and magnetic microbeads. Color microspheres include color polymer microspheres and colloidal gold solution.

6. The detection assay of claim 1, wherein both the chemiluminescent conjugate and the capture agent include a immunoconjugate, comprising antibodies, antigens, biotins, avidin and their analogues. For the avidin analogues, straptavidin is the most common choice for this detection.

7. The detection assay of claim 1, wherein the chemiluminescent label used for the labeling of the immunoconjugates can either be the direct luminescent labels as acridine ester and acridine sulfonamide, enzymic catalyzed luminescent labels as horseradish peroxidase and alkaline phosphatase, or the electrochemiluminescent label as tripyridine ruthenium.

8. The detection assay of claim 1, wherein the chemiluminescence reaction solution includes a direct luminescence reaction solution containing hydrogen peroxide in an alkaline state, an enzymatic luminescence reaction solution in which luminol and its derivatives are luminescent substrates, and an electrochemiluminescence on the electrodes in a ruthenium terpyridine structural labels solution.

9. The detection assay of claim 1, wherein the chemiluminescent conjugate is in the form of lyophilized powder.

10. The detection assay of claim 1, wherein the solid membrane is provided with a blood cell separation structure with direct connection at the proximal side, and the blood cell separation structure includes a blood cell separation membrane pad or a membrane pad treated with the antibody against red blood cells.

11. The detection assay of claim 1, wherein the solid membrane comes with a liquid dispersion membrane pad with direct connection at the proximal side, wherein the liquid dispersion membrane pad includes a glass fiber membrane pad or a polyester fiber membrane pad.

12. The detection assay of claim 1, wherein the detection assay of the testing strip comprises a joint combination of biotin/avidin detection system, therefore the sample mixture includes the chemiluminescent label labeled primary immunoconjugate, a biotin labeled secondary immunoconjugate and the testing sample, and the solid membrane coated with unlabeled avidin and its analogues as the capture agent in the detection.

13. The detection assay of claim 1, wherein the detection assay comes with a chemiluminescent reader, which is able to quantitatively detect the amount of light being induce to emit from the solid membrane.

14. The detection assay of claim 1, wherein the operation of the membrane based chemiluminescence immunochromatographic detection assay includes the following steps:
1) take the solid membrane structure coated with the capture agent in the scattered, distributed manner, connect the liquid dispersion membrane and/or blood cell separationstructure at the proximal side in turn, connect the water absorption structure at the distal side then, and place it at a level position;
2) take the sample, add it into a tube with the chemiluminescent conjugate, take the test solution and add it into the tube again, mix it, and form the sample mixture to be tested;
3) add the sample mixture onto the liquid dispersion membrane and allow it to flow forward through the blood cell separationstructure and the solid membrane, and is absorbed in the water absorbent pad;
4) take and add the testing buffer onto the liquid dispersion membrane and allow it to flow forward through the blood cell separationstructure and the solid membrane, and is absorbed in the water absorbent pad;
5) take the solid membrane, transfer it to the chemiluminescence reaction solution and the chemiluminescence reader for the quantitative detection of the amount of light from the solid membrane;
6) calculate the concentration of the target analyte in the testing sample based on the standard curve and complete the test.

15. An application of the membrane based chemiluminescence immunochromatographic detection assay in the development of immunoassay reagent products.
